(19) United States
(12) Patent Application Publication
(10) Pub. No.: US 2008/0299060 A1
(43) Pub. Date: Dec. 4, 2008

(54) Pharmaceutical Composition for the Treatment of Nail Diseases

(75) Inventors: Alfredo Bruno, Biel-Benken (CH); Christian Frei, Basel (CH); Werner Henrich, Binningen (CH)

Correspondence Address: DICKSTEIN SHAPIRO LLP, 1177 AVENUE OF THE AMERICAS (6TH AVENUE), NEW YORK, NY, 10036-2714, US

(73) Assignee: TLT MEDICAL LTD., Reinach (CH)

(21) Appl. No.: 12/159,921

(22) PCT Filed: Dec. 29, 2006
(86) PCT No.: PCT/CH06/00741

§ 371 Date: Jul. 2, 2008

(30) Foreign Application Priority Data
Jan. 2, 2006 (EP) 06000001.5

(51) Int. Cl. A61K 8/40; A61K 31/135

(52) U.S. Cl. 424/61; 514/649

(57) ABSTRACT

A method for topically and systemically delivering a pharmaceutical composition to a human nail is disclosed, said method comprising the application of a pharmaceutical composition to the nail, covering the nail and the pharmaceutical composition with a protective layer, and a dissolvent to remove the protective layer and the pharmaceutical composition after a certain time of action and before reapplication. Preferably the method is combined with a laser pretreatment of the nail to drill arrays of partial orifices to enhance the permeability of drugs through the nail plate.

The present invention relates to a method to cure, ameliorate or prevent nail diseases and non-nail diseases upon application of a pharmaceutical composition containing at least a therapeutically active substance to treat the disease in question.

The human nail plate is thick, hard, dense, and represents a barrier for drugs to penetrate down to the nail bed in a quantity capable of inducing a therapeutical action. The nail material is similar to the stratum corneum of the skin, being derived from epidermis, and is composed primarily of hard keratin, which is highly disulfide-linked, and is approximately 100-fold thicker than stratum corneum. In order to deliver therapeutically significant amounts of drugs into and across the nail plate, the permeability of the nail plate to the drug used must be enhanced by chemical or mechanical methods.

U.S. Pat. No. 6,231,875 describes a method for topical treatment of nail and skin diseases. The patent relates to an acidified composition and methods for increasing the permeability of a nail plate by means of topically applying an acidified composition to the nail plate. U.S. Pat. No. 5,972,317 describes a method for treating diseased nails by topically applying a nail-permeable composition to the nail plate which contains a proteolytic enzyme and a medicament. U.S. Pat. No. 5,181,914 describes a medicating device for human diseased nails and adjacent tissue which contains a viscoelastic gel pad.

The patent application WO 2004084826 describes a method of ameliorating or preventing fungal infection of the nails. Composition embodiments in the form of one-coat type and two-coat type suitable for daily fungicidal regimens are disclosed. A preferred antifungal nail coat composition comprises an effective fungicidal amount of antifungal agent, a permeation enhancing amount of a substantially non-volatile permeation enhancer, a film-forming amount of a hydrophilic polymer, and a pharmaceutically acceptable, volatile carrier. The composition provides a substantially water-soluble fungicidal coating in contact with a fungally susceptible or infected nail.

According to the WO 0211764 it has been found that nail diseases like onychomycosis can be successfully treated by forming with a laser one or more small orifices into a nail plate, and applying an antifungal containing composition to the nail in order to secure a sufficient penetration of the drug into the deeper layers of the nail and to the nail bed. An orifice described in that patent application means any small orifice or depression that penetrates 80 to 100% of the nail plate. According to WO 03068197 not only nail diseases but other diseases such as e.g. arthritis can be treated by forming with a laser one or more small orifices into the nail plate and applying to the pretreated nail a composition containing a therapeutically active substance for the disease in question which permeates through the nail and enters the blood stream.

The present invention provides an improved method for delivering a therapeutically active substance to the nail, the nail bed or to the blood for its systemic redistribution in the neighboring tissue or in the whole human body. This method, which provides a higher permeation of therapeutically active substances through the nail than the methods of the prior art results thus in higher efficacy, comprises:
a) Applying to the nail a pharmaceutical composition containing a therapeutically active substance and,
b) covering the nail with a protective layer and,
c) before reapplication of the pharmaceutical composition and the protective layer, re-dissolving the protective layer with a dissolvent containing a therapeutically active substance.

The pharmaceutical composition may be in the form of a liquid, a semi-solid, a solution, a gel, a cream or an emulsion. Considering that the pharmaceutical composition is intended for multiple applications, e.g. once or twice daily or once a week, prior to a new application the protective layer is re-dissolved and partially cleaned with a dissolvent which contains the same therapeutically active substance as in the pharmaceutical composition or another therapeutically active substance. The dissolvent in the pharmaceutical composition is able to dissolve the protective layer. In a preferred embodiment the dissolvent and the pharmaceutical composition are identical.

Although the present method works in untreated nails as well, it is most suitable for pre-treated nails e.g. with arrays of equally spaced partial holes drilled with a near infrared pulsed laser as described in WO 0211764. The array of holes functions as a depot of the pharmaceutical composition while a protective layer subsequently applied prevents the volatile solvents of the pharmaceutical composition to evaporate into the air. In this way, the solvents, acting as vehicles for the therapeutically active substance, are forced to penetrate into the keratin tissue of the nail. In addition, by the application of a protective layer precipitation of the active substance on the orifices is minimized, the pharmaceutical composition is prevented to flow out of the orifice and dirt and germs as e.g. bacteria are inhibited from entering the orifices.

Following administration of the pharmaceutical composition to the nail, a continuous protective layer is placed on the outer surface of any treated nail. The protective layer covers also the orifices in the case of laser pretreated nails according to WO 0211764. Examples of materials useful to form a protective layer include, but are not limited to, nail varnishes, nail polishes, nail lacquers, film forming solutions or sprays. Such compositions may contain the same therapeutically active substance as in the pharmaceutical composition or another therapeutically active substance or they may not contain a therapeutically active substance. Preferably the same active substance as in the pharmaceutical composition is contained in the protective layer.

According to the present invention, a dissolvent is used to remove an existing protective layer before reapplication of the pharmaceutical composition and the protective layer. Such a dissolvent can have different functions on the nail plate, including:
Dissolution of previously applied pharmaceutical compositions and protective layers to allow the application of a new dosage.
Cleaning the nail before the application of the pharmaceutical composition or the protective coating, where the dirt or debris might stem from the nail pretreatment by e.g. mechanical means, photo-ablation or by chemical etching methods. Cleaning may include, besides the dissolvent, the use of cleaning instruments such as brushes, cotton swabs, cotton pads or other means.
Disinfection of the nail plate.
Chemical preparation of the nail plate for better permeation of subsequently applied pharmaceutical compositions and their active ingredients to the nail bed.

Suitable solvents for the pharmaceutical composition can be aliphatic and aromatic alcohols, sulfoxides, fatty acids, fatty acid esters, polyols, amides, surfactants, terpenes, alkanones, organic acids and mixtures thereof.

Suitable alcohols include, without limitation, ethanol, propanol, butanol, pentanol, hexanol, octanol, nonanol, decanol, 2-butanol, 2-pentanol, benzyl alcohol, phenoxyethanol, caprylic alcohol, decyl alcohol, lauryl alcohol, 2-lauryl alcohol, myristyl alcohol, cetyl alcohol, stearyl alcohol, oleyl alcohol, linolyl alcohol, linolenyl alcohol and mixtures thereof. Volatile aliphatic alcohols having 2 to about 5 carbon atoms can provide a dual function of serving both as volatile carrier and penetration enhancer. The aromatic alcohols, such as benzyl alcohol, phenoxyethanol, and the like can provide a dual function of serving both as a substantially non-volatile, permeation enhancer and auxiliary anti-infective. Preferred alcohols are ethanol and benzyl alcohol.

Suitable sulfoxides include dimethylsulfoxide, decylmethylsulfoxide, and mixtures thereof.

Suitable fatty acids include valeric, heptanoic, pelargonic, caproic, capric, lauric, myristic, stearic, oleic, linoleic, linolenic, caprylic, isovaleric, neopentanoic, neoheptanoic, neononanoic, trimethyl hexanoic, neodecanoic and isostearic acids, and mixtures thereof.

Suitable fatty acid esters include isopropyl n-butyrate, isopropyl n-hexanoate, isopropyl n-decanoate, isopropyl myristate, isopropyl palmitate, octyidodecyl myristate, ethyl acetate, butyl acetate, methyl acetate, methylvalerate, methylpropionate, diethyl sebacate, ethyl oleate, ethyl laurate and mixtures thereof.

Suitable polyols include propylene glycol, polyethylene glycol, ethylene glycol, diethylene glycol, triethylene glycol, dipropylene glycol, glycerol, propanediol, sorbitol, dextrans, butanediol, pentanediol, hexanetriol, and mixtures thereof.

Suitable amides include urea, dimethylacetamide, diethyltoluamide, dimethylformamide, dimethyloctamide, dimethyldecamide, pyrrolidone derivatives, 1-alkyl-4-imidazolin-2-one, cyclic amides, hexamethylenelauramide and its derivatives, diethanolamine, triethanolamine and mixtures thereof. Suitable pyrrolidone derivatives includel-methyl-2-pyrrolidone, 2-pyrrolidone, 1-lauryl-2-pyrrolidone, 1-lauryl-4-carboxy-2-pyrrolidone, 1-methyl-4-carboxy-2-pyrrolidone, 1-hexyl-4-carboxy-2-pyrrolidone, 1-decylthioethyl-2-pyrrolidone, N-cyclohexyl-pyrrolidone, 1-methyl-4-methoxycarbonyl-2-pyrrolidone, 1-hexyl-4-methoxy-carbonyl-2-pyrrolidone, 1-lauryl-4-methoxycarbonyl-2-pyrrolidone, N-dimethylamino-propyl-pyrrolidone, N-cocoylpyrrolidone, N-tallowylpyrrolidone, fatty acid esters of N-(2-hydroxymethyl)-2-pyrrolidone, and mixtures thereof. Suitable cyclic amides include, 1-dodecylazacycloheptan-2-one, 1-geranylazacycloheptan-2-one, 1-farnesylazacycloheptan-2-one, 1-geranyl-geranyl-azacycloheptan-2-one, 1-(3,7-dimethyloctyl) azacycloheptan-2-one, 1-(3,7,11-trimethyl-octyl) azacycloheptan-2-one, 1-geranylazacyclohexan-2-one, 1-geranyl-azacyclopentan-2,5-dione, 1-farnesylazacyclopentan-2-one, and mixtures thereof.

Suitable surfactants include anionic surfactants, cationic surfactants, nonionic surfactants, amphoteric surfactants and lecithin. Suitable anionic surfactants include sodium laurate, sodium lauryl sulfate, and mixtures thereof.

Suitable cationic surfactants include cetyltrimethylammonium bromide, tetradecyltrimethyl ammonium bromide, benzalkonium chloride, octadecyltrimethyl ammonium chloride, cetylpyridinium chloride, dodecyltrimethylammonium chloride, hexadecyltrimethylammonium chloride, and mixtures thereof. Suitable nonionic surfactants include alpha-hydro-(D-hydroxy poly(oxyethylene)-poly(oxypropyl) poly(oxyethylene) block copolymers, polyoxyethylene ethers, polyoxyethylene sorbitan esters, polyethylene glycol esters of fatty alcohols, and mixtures thereof. Suitable alpha-hydro-co-hydroxy-poly(oxyethylene)-poly(oxypropyl) poly (oxyethylene) block copolymers include Poloxamers 182, 184, 231, and mixtures thereof. Suitable polyethylene glycol esters of fatty acids include polyoxyethylene, polyoxyethylene monostearate, the polyoxyethylene monostearate and mixtures thereof.

Suitable amphoteric surfactants include, without limitation thereto, lauramidopropyl betaine, cocamidopropyl betaine, lauryl betaine, cocobetaine, cocamidopropyl-hydroxy-sultaine, aminopropyl laurylglutamide, sodium cocoamphoacetate, sodium lauro-amphoacetate, disodium lauroamphodiacetate, disodium cocoamphodiacetate, sodium-cocoamphopropionate, disodium lauroamphodipropionate, disodium cocoampho-dipropionate, sodium-lauriminodipropionate, disodium-cocoampho-carboxy-methylhydroxy-propylsulfate, and the like.

Suitable terpenes include D-limonene, α-pinene, β-enrene, α-terpineol, terpinen-4-ol, carvol, carvone, pulegone, piperitone, menthon, menthol, geraniol, cyclohexene oxide, limonene oxide, α-pinne oxide, cyclopentene oxide, 1,8-cineol, ylang ylang oil, anise oil, chenopodium oil, eucalyptus oil, and mixtures thereof. Suitable alkanones include N-heptane, N-octane, N-nonane, N-decane, N-undecane, N-dodecane, N-tridecane, N-tetradecane, N-hexadecane, and mixtures thereof. Suitable organic acids include citric acid, succinic acid, salicylic acid, salicylates (including the methyl, ethyl and propyl glycol derivatives), tartaric acid, and mixtures thereof.

A preferred composition for the protective layer will contain a poorly water-soluble or water-insoluble film-forming polymer to prevent that the pharmaceutical composition is removed upon washing. There is no limitation on the amount of polymer used in the composition provided that the nail coat composition can be easily applied to the nail and have good adhesive properties and sufficient abrasive resistance to form a film on the dorsal surface of the nail plate.

A possible variant of the protective layer comprises similar amounts of monobutyl ester of poly-methyl-vinyl ether-maleic acid (e.g. Gantrez® ES-435, ISP USA), ethylene acetate and ethanol. A preferred variant of the protective layer will comprise 33.3% Gantrez® ES-435, 33.3% ethyl acetate and 33.3% ethanol where the percentage refers to the weight of the component in relation to the total weight of the composition, i.e. w/w. In a preferred embodiment, where the therapeutically active substance is an antifungal agent, the composition to form the protective layer is composed of equal amounts of ethyl acetate, ethanol, and Gantrez® ES-435, and 5 to 15% w/w of terbinafine of the total mass of the protective layer composition.

The pharmaceutical composition used in the method of the invention comprises at least one therapeutically active substance which may include, without limitation, photosensitizers, androgens, estrogens, nonsteroidal anti-inflammatory agents, antihypertensive agents, analgesic agents, antidepressants, antibiotics, anticancer agents, anesthetics, antiemetics, antiinfectants, contraceptives, antidiabetic agents, steroids, anti-allergy agents, anti-migraine agents, agents for smoking cessation, anti-obesity agents, antifungal agents and anti-psoriatic agents.

For the case where the disease is onychomycosis, the preferred therapeutically active substance in the pharmaceutical composition and in the protective layer is terbinafine. Terbinafine is a proven antifungal agent (sold under the brand name Lamisil®) applied in the present method of invention at concentrations of 1 to 20% w/w but most preferably at a concentration between 5 and 15% w/w.

For the case where the disease is nail psoriasis, the therapeutically active substance in the pharmaceutical composition and in the protective layer could include, but is not limited to, corticosteroids, intralesional corticosteroids, fluorouracil, calcipotriol or anthralin tazarotene. Potent corticosteroids such as clobetasole or beclomethasone dipropionate or halobetasol could be appropriate. Medium-potency agents such as triamcinolone (Aureocort, or tri-Adcortl) or the vitamine D3 topical agent, calcipotriene, may be equally beneficial in a high frequency dose treatment.

Examples of therapeutically active substances include the following substances: acebutolol, acetylcysteine, acetaminophen, acetylsalicylic acid, acyclovir, alprazolam, alfacalcidol, allantoin, allopurinol, aloe vera, ambroxol, amikacin, amiloride, aminoacetic acid, amiodarone, amitriptyline, amlodipine, amoxicillin, ampicillin, anthralin, ascorbic acid, astemizole, atenolol, beclomethasone dipropionate, bee propolis, benserazide, benzalkonium hydrochloride, benzocaine, betamethasone, bezafibrate, biotin, biperiden, bisoprolol, bromazepam, bromhexine, bromocriptine, budesonide, bufexamac, buflomedil, bupivacaine, buspirone, caffeine, calcipotriene, calcipotriol, camphor, captopril, carbamazepine, carbidopa, carboplatin, cefachlor, cefalexin, cefatroxil, cefazolin, cefixime, cefotaxime, ceftazidime, ceftriaxone, cefuroxime, selegiline, chloramphenicol, chlorhexidine, chlor-pheniramine, chlortalidone, choline, cyclosporin, cilastatin, cimetidine, ciprofloxacin, cisapride, cisplatin, clarithromycin, clavulanic acid, clomidine, clomipramine, clonazepam, clonidine, clotrimazole, codeine, cholestyramine, clobetasole, cromoglycic acid, cyanocobalamin, cyclosporine, cyproterone, desogestrel, dexamethasone, dexpanthenol, dexamethasone, dextromethorphan, dextropropoxiphen, diazepam, diclofenac, digoxin, dihydrocodeine, dihydroergotamine, dihydroergotoxin, diltiazem, diphenhydramine, dipyridamole, dipyrone, disopyramide, domperidone, dopamine, doxycycline, enalapril, ephedrine, epinephrine, ergocalciferol, ergotamine, erythromycin, estradiol, ethinylestradiol, etoposide, Eucalyptus globulus, famotidine, felodipine, fenofibrate, fenoterol, fentanyl, flavin mononucleotide, fluconazole, flunarizine, fluorouracil, fluoxetine, flurbiprofen, folic acid, folinic acid, furosemide, gallopamil, gemfibrozil, gentamicin, Gingko biloba, glibenclamide, glipizide, clozapine, Glycyrrhiza glabra, griseofulvin, halobetasol, haloperidol, heparin, hyaluronic acid, hydrochlorothiazide, hydrocodone, hydrocortisone, hydromorphone, ipratropium hydroxide, ibuprofen, imipenem, indomethacin, insulin, iohexol, iopamidol, isosorbide dinitrate, isosorbide mononitrate, isotretinoin, ketotifen, ketoconazole, ketoprofen, ketorolac, labetalol, lactulose, levocarnitine, levodopa, levoglutamide, levonorgestrel, levothyroxine, lidocaine, lipase, imipramine, lisinopril, loperamide, lorazepam, lovastatin, medroxyprogesterone, menthol, methotrexate, methyldopa, methylprednisolone, methyltestosterone, metoclopramide, metoprolol, methotrexate, miconazole, midazolam, minocycline, minoxidil, misoprostol, morphine, multivitamin mixtures and combinations and mineral salts, N-methylephedrine, naftidrofuryl, naproxen, neomycin, nicardipine, nicergoline, nicotinamide, nicotine, nicotinic acid, nifedipine, nimodipine, nitrazepam, nitrendipine, nitroglycerine, nizatidine, norethisterone, norfloxacin, norgestrel, nortriptyline, nystatin, ofloxacin, omeprazole, ondansetron, pancreatin, panthenol, pantothenic acid, paracetamol, penicillin G, penicillin V, phenobarbital, pentoxifylline, phenoxymethylpenicillin, phenylephrine, phenylpropanolamine, phenytoin, piroxicam, polymyxin B, povidone-iodine, pravastatin, prazepam, prazosin, prednisolone, prednisone, prilocaine, progesterone, propafenone, propranolol, proxyphylline, pseudoephedrine, pyridoxine, quinidine, ramipril, ranitidine, reserpine, retinol, retinoids, riboflavin, rifampicin, rutoside, salbutamol, salcatonin, salicylic acid, scopolamine, simvastatin, somatotropin, sotalol, spironolactone, sucralfate, sufentanil, sulbactam, sulfamethoxazole, sulfasalazine, sulpiride, sumatriptan, tamoxifen, tazarotene, tegafur, teprenone, terazosin, terbinafine, terbutaline, terfenadine, testosterone, tetracaine, tetracycline, theophylline, thiamine, ticlopidine, timolol, tranexamic acid, tretinoin, triamcinolone acetonide, triamterene, trimethoprim, troxerutin, uracil, valproic acid, vancomycin, verapamil, vitamin A, vitamin C, vitamin E, and zidovudine. A combination of active ingredients may also be used.

In another aspect the therapeutically active substance of the pharmaceutical composition of the invention may comprise a vaccine. Vaccines may include without limitation Smallpox, Rabies, Plaque, Diphteria, Pertussis, Tuberculosis, Tetanus, Yellow Fever, Injectable Polio Vaccine, Oral Polio Vaccine, Measales, Mumps, Rubella, Hepatitis B, Hepatitis C, Haemophilus influenza Typ B, Japanese Encephalitis, Biomanguinhos, Human Influenza Typ B (Hib), HIV, cancer.

The amount of therapeutically active substance in the pharmaceutical composition may vary from 0.1 weight percent to 100 weight percent based on the total weight of the pharmaceutical composition. Preferably the therapeutically active substance is present in an amount of from 0.1% to 99%, preferably from 1% to 20%, more preferably 5% to 15%, weight percent, based on the total weight of the pharmaceutical composition. The dose of the therapeutically active substance depends to a certain extent, in untreated nails, on the applied mass of the pharmaceutical composition, which is limited by the visco-elastic flow properties of the composition and the area of the nail plate. In laser pre-treated nails with orifices, the dose of the therapeutically active substance depends additionally on the number, the volume, the diameter and the shape of the orifices. The exposure time of the therapeutically active substance in this case depends on the number, diameter, shape and depth of the orifices and on the sealing properties of the protective layer. The required dose and dose regime of the pharmaceutical composition have to be determined according to the nature and severeness of the disease to be treated.

Suitable dissolvents for the removal of the protective layer prior to reapplication can be the same solvents as used for the pharmaceutical composition listed above including: aliphatic and aromatic alcohols, sulfoxides, fatty acids, fatty acid esters, polyols, amides, surfactants, terpenes, alkanones, organic acids and mixtures thereof. The preferred dissolvent is ethanol.

Additional ingredients may be used in the pharmaceutical compositions or applied directly to the treated or untreated nail prior to or following the application of the pharmaceutical composition to the nails. Such additional ingredients include natural and/or artificial ingredients which are commonly used to prepare pharmaceutical compositions. Examples of additional ingredients include surfactants, binders, disintegrating agents, vitamins, botanicals, supplements, herbs, minerals, trace elements, amino acids, fibers, enzymes, fillers, buffers, colorants, dyes, antioxidants, preservatives, electrolytes, glidants, disintegrates and lubricants. A combination of additional ingredients, known to those skilled in the art, may also be used.

In one aspect the method of the invention provides a controlled delayed release, e.g. sustained and prolonged release of the therapeutically active substance from the pharmaceutical composition that may be used for the continuous treatment of diseases over a period of time.

In a further aspect, the method of the invention provides a controlled fast release e.g. an immediate release of the therapeutically active substance from the pharmaceutical composition. The fast release from the pharmaceutical composition may be used to administer the therapeutically active substance systemically and to avoid a first path effect that may occur by oral administration. The delivery of the therapeutically active substance from the pharmaceutical composition through the orifices in laser pretreated nails allows the administration of the therapeutically active substance directly to the well-perfused nail bed where it enters the blood-stream.

When the method is used in laser pretreated nails numerous orifices are formed in the nail, and the orifices may traverse the entire nail or the nail is only partially etched or drilled depending on the desired mode of treatment and strength of the pharmaceutical composition. The diameter of the orifice is preferably from 1 μm (μm=micron) to 1 mm, more preferably from 100 μm to 400 μm, most preferably from 200 μm to 350 μm. The orifices are preferably of cylindrical or conical shape.

Typically up to about 500 orifices per cm², where the orifices are partial or full orifices, may be formed in the nail, more preferably 400 orifices per cm². In this way about 200 up to about 2,000 orifices are formed in a typical toenail, but most typically, about 600 orifices in a great toenail.

Any laser type may be used to produce the orifices provided it is capable of inducing efficient photoablation on the nail tissue such as excimer, Er:YAG, Ho:YAG, or CO₂ lasers. Photoablation is achieved by pulsed laser irradiation of a selected wavelength, power and pulse duration according to the thermal, mechanical and spectral characteristics of the tissue of interest. The deposited electromagnetic energy is almost entirely transformed into mechanical energy (i.e. hv≈mv²/2) and the illuminated region is ejected in the form of debris escaping the orifice at ca. 1,000 m/s. In a preferred photoablation process, as the debris removes the deposited energy, the irradiated nail is not heated thus minimizing discomfort.

The pharmaceutical composition, protective layer and dissolvent used in the method of invention could be made available for commercial purposes in the form of a kit including also brushes or other devices to be used for the application and/or cleaning of the nail before the first applications and also for subsequent applications.

A preferred embodiment of an application kit comprises:
I) A bottle with a pharmaceutical composition which contains a vehicle able to dissolve the protective layer,
II) A second bottle filled with a substance to form a protecting layer over the nail plate after application of the pharmaceutical composition and, optionally,
III) Brushes and other devices needed for the application of the pharmaceutical composition (I), cleaning of the nails and removal from the protective layer (II).

Another embodiment of an application kit comprises a box containing:
i) A bottle with a pharmaceutical composition which contains a vehicle able to dissolve the protective layer,
ii) A second bottle filled with a substance to form a protecting layer over the nail plate after application of the pharmaceutical composition,
iii) A third bottle with the dissolvent to clean the nail before the subsequent application of the pharmaceutical composition and the protective layer and, optionally,
iv) Brushes and other devices needed for the application of the pharmaceutical composition (i), cleaning of the nails and removal from the protective layer (ii).

Preferentially the bottles of the pharmaceutical composition, the substance to form a protective layer and the dissolvent have twist caps with an application brush to transfer the compositions from the bottles to the nail plate as usually used in nail polish products. The fibers of the application brush could have a length between 3 and 20 mm, preferentially between 8 and 15 mm, and the diameters of the fibers are 50 to 10 μm, preferentially between 50 and 150 μm. The fibers of the cap brush are assembled in a tight bundle of 1 to 5 mm in diameter.

The fibers of the cleaning brush (III and iv) of the application kit could have a length between 3 and 20 mm, preferentially between 8 and 10 mm, and the fibers diameter are between 10 and 400 μm, preferentially between 50 and 150 μm. As a variation of the concept also conical fibers with pointed tips can be applied. Another variation is a brush with fibers of different lengths. The fibers of the cleaning brush can be fixed on the brush head as single fibers, in bundles with few fibers, in tight bundles of many fibers or in one single tight fiber bundle. The fiber bundles can have diameters between 0.1 and 2 mm.

The cleaning brush could also be electrically powered to vibrate as those used nowadays to brush teeth where the brush is a disposable part of an electrical larger device. In this way the cleaning is more effective and faster and precludes the formation of tiny bubbles in the orifices when used in pretreated nails.

The box of the application kit should preferentially be made of a material which is not sensitive to humidity and which prevents light entering the box when closed.

The virtue of the invention is illustrated in the following example.

### EXAMPLE

### In-vitro Nail Permeation Assay of an Antifungal Agent

Permeation fluxes of active substances, determined from nail permeation assays, are used to predict the efficacy of the active substances in a clinical situation. A permeation assay similar to the one reported by Franz (T. J. Franz, Dermatology, 184, 18-20, 1992) was used to investigate the applicability of the present invention to the treatment of onychomycosis and, in particular, to evaluate the permeation fluxes of a terbinafine containing pharmaceutical composition and a protective layer in laser pre-treated human cadaver great toenails. The experimental setup emulates the clinical situation where the pharmaceutical composition remains liquid for an extended period of time inside the nail holes in the case of laser pretreated nails to maximize the diffusion of the terbinafine into the nail.

Human cadaver great toenails selected for the tests were soaked in physiological buffer solution for several hours before starting the assay. Nails were prepared for the assay by removal of epidermal residues on the ventral side. Round nail discs of 16 mm in diameter were punched. For the laser pretreated nails, the perforated area had a diameter of 13 mm. Nails were masked on the dorsal side by a silicon mounting ring leaving a nail area of 10 mm in diameter where the nails were exposed to the pharmaceutical composition and the protective layer.

Three groups of nail samples were investigated. They are indicated in Table 1:

Group 1: Untreated nail samples were used in this group which is the control group. These nails were exposed to 25 μL of a lacquer containing 9% w/w terbinafine once a day.

In the other two groups, nail samples perforated by an Er:YAG laser with 400 holes /cm² at a depth of 50-80% of their full thickness were used to demonstrate the superiority of the present invention with respect to the prior art.

Group 2: In this group perforated nails were used. These nails were exposed to 25 μL of a 10% w/w terbinafine in ethanol solution once a day. After the application of the 10% w/w ethanolic solution to these nails, nails were covered with 25 μL of a lacquer containing 9% w/w terbinafine to form the protective layer according to the present invention.

Group 3: In this group perforated nails were used. These nails were exposed to 25 μL of a 10% w/w terbinafine in ethanol solution once a day. After the application of the 10% w/w ethanolic solution to these nails, these nails were covered with 25 μL of a lacquer without terbinafine to form the protective layer according to the present invention.

Franz type diffusion cells made of glass were used in the assays having a volume of ca. 5 mL using demineralized water as receptor buffer. Nail samples were fixed on the Franz cells with sealing silicone and Teflon discs pressed with a metallic spring loading system. In this way the ventral sides of the nails were in contact with demineralized water in the cell while the dorsal sides were in contact with the pharmaceutical formulations and protective layer according to Table 1. Diffusion cells were kept at 32° C. during the permeation assays.

Cleaning of the nail sample and dissolution of previous layers of pharmaceutical composition and protective layers were performed prior to every reapplication of the pharmaceutical composition and the protective layer. For this purpose 100 microliters of dissolvent were applied to every nail sample. Subsequently, the nail samples were rubbed with a cleaning brush to ensure an exhaustive cleaning and then were dried with a cotton swab to remove with the dissolvent also the old components of the pharmaceutical solution and the protective layer. 25 microliters of the pharmaceutical composition were then applied to the exposed nail area and distributed with the application brush.

TABLE 1
Nail groups investigated. Dose regime was once a day for the three groups.

| | | Pharmaceutical | Protective | |
|---|---|---|---|---|
| Group | Nail type | composition | layer | Dissolvent |
| Group 1 | Untreated nails | 9% w/w terbinafine | The protective layer | Ethanol |
| | | lacquer | is the lacquer | |
| Group 2 | Treated nails¹ | 10% w/w terbinafine in | 9% w/w terbinafine | 10% w/w terbinafine |
| | | ethanol | lacquer | in ethanol² |
| Group 3 | Treated nails¹ | 10% w/w terbinafine in | Lacquer without | 10% w/w terbinafine |
| | | ethanol | terbinafine | in ethanol² |

¹Laser pretreated nails with 400 partial orifices/cm² drilled as described in text.
²Also used to clean the nail samples after laser pretreatment.
³w/w refers to the ratio of weight of terbinafine hydrochloride salt to the total weight of vehicle or ethanolic solution.

Samples of receptor fluid were collected at various time points and analyzed by liquid chromatography and mass spectroscopy. The removed sample volume was replaced by demineralized water. This dilution effects were accounted in the final determination of the permeation fluxes.

The mean values of the concentration of terbinafine in the Franz cells, given in ng/mL, at days 3 and 5 are reported in table 2 together with their standard deviations for the three groups investigated. The permeation fluxes calculated with these data are also reported in table 2. These mean permeation fluxes correspond to a 34-fold increase in the permeation fluxes as compared to the control group (group 1) using the disclosed embodiment in which the protective layer also contains terbinafine (group 2), and a 5-fold increase as compared to the control group (group 1) in the disclosed embodiment in which the protective layer does not contain terbinafine (group 3). These results strongly suggest that the topical application of terbinafine as active substance in the pharmaceutical composition to treat onychomycosis using the disclosed method should result in higher efficacies and/or shorter treatment durations than in therapies using prior art methods.

TABLE 2
Mean values of permeation fluxes of terbinafine concentrations
measured at days 3 and 5 in the 5 mL receptor chambers of the
Franz cells for each group investigated.

| | Day 3 | Day 5 | |
|---|---|---|---|
| | Mean value | Mean value | |
| Group | in ng/mL | in ng/mL | Permeation |
| (Number of | (Standard | (Standard | Fluxes in |
| nails) | deviation) | deviation) | ng/cm²/h |
| Group 1 | 82.4 | 115.1 | 4.34 |
| (3) | (68.5) | (67.0) | |
| Group 2 | 305.2 | 1432.9 | 149.6 |
| (3) | (330.6) | (895.4) | |
| Group 3 | 94.5 | 272.6 | 23.6 |
| (3) | (129.2) | (124.8) | |

1. A method of treating a disease comprising a) applying to a nail a pharmaceutical composition containing a therapeutically active substance and b) covering the nail with a protective layer c) dissolving the protective layer with a dissolvent containing a therapeutically active substance and, reapplying the pharmaceutical composition and the protective layer.

2. Method according to claim 1 where the pharmaceutical composition is a solution, gel or cream.

3. Method according to claim 1 where the protective layer is a lacquer, a varnish or a nail polish.

4. Method according to claim 1 where the protective layer contains the same therapeutically active substance as the pharmaceutical composition.

5. Method according to claim 1 where the dissolvent contains the same therapeutically active substance as the protective layer and the pharmaceutical composition.

6. Method according to claim 1 where the pharmaceutical composition is an ethanolic solution of the therapeutically active substance.

7. Method according to claim 1 where the protective layer, in addition to the therapeutically active substance, contains a water-insoluble or poorly water-soluble polymer.

8. Method according to claim 1 where the nail is pretreated to contain full or partial holes.

9. Method according to where the disease is a nail disease.

10. Method according to claim 1 where the disease is not a nail disease.

11. Method according to claim 9 where the therapeutically active substance is terbinafine.

12. Kit for the treatment of a disease comprising. a) A first recipient containing a composition providing after its application a protective layer over the nail and b) a second recipient containing a therapeutically active substance and a vehicle able to dissolve the protective layer.

13. Kit according to claim 12 where a) contains a therapeutically active substance.

14. Kit according to claim 13 where a) and b) contain the same therapeutically active substance.

15. Kit according to claim 12 where the therapeutically active substance is terbinafine.

16. Kit according to claim 15 where a) is an ethanolic terbinafine solution and b) is a terbinafine containing lacquer.

17. Kit for the treatment of a disease comprising a) A first recipient containing a substance providing after application a protective layer over the nail and, b) a second recipient containing a therapeutically active substance and c) a third recipient containing a dissolvent for the protective layer.

18. Kit according to claim 17 where a) contains a therapeutically active substance.

19. Kit according to claim 18 where c) contains a therapeutically active substance.

20. Kit according to claim 12 containing a mechanical cleaner adapted to remove the protective layer from the nail in combination with the dissolvent.

21. Kit according to claim 20 containing a brush.

22. Kit according to claim 21 where the brush is a vibrating brush.
